(19) United States
(12) Patent Application Publication
(10) Pub. No.: US 2026/0115625 A1
(43) Pub. Date: Apr. 30, 2026

(54) MEMBRANE CONTACTOR BASED LIQUID-LIQUID EXTRACTION PROCESS FOR BIOFUEL SEPARATION

(71) Applicant: UT-Battelle, LLC, Oak Ridge, TN (US)

(72) Inventors: Syed Z. Islam, Oak Ridge, TN (US); Ramesh R. Bhave, Knoxville, TN (US); Shailesh Dangwal, Oak Ridge, TN (US); Blake Trusty, Oak Ridge, TN (US)

(21) Appl. No.: 19/371,360

(22) Filed: Oct. 28, 2025

Related U.S. Application Data
(60) Provisional application No. 63/713,253, filed on Oct. 29, 2024.

(51) Int. Cl. B01D 11/04; B01D 1/22; B01D 69/02; B01D 69/08; B01D 71/26; B01D 71/34; C12P 7/18

(52) U.S. Cl. CPC B01D 11/0415; B01D 1/22; B01D 11/0492; B01D 69/02; B01D 69/08; B01D 71/262; B01D 71/34; C12P 7/18; B01D 2325/02833; B01D 2325/04; B01D 2325/36

(57) ABSTRACT

The present invention provides a method for the recovery of 2,3-butanediol (BDO) from an aqueous fermentation broth. The method includes directing an aqueous phase along the lumen side or the shell side of a plurality of hydrophobic hollow fibers while simultaneously directing an organic phase along the other of the lumen side or the shell side of the plurality of hollow fibers. The aqueous phase includes a fermentation broth containing BDO, water, and impurities, and the organic phase is initially composed entirely of an organic solvent, the organic solvent being immiscible in water and having an affinity for BDO. The organic solvent continuously recovers BDO from the fermentation broth, the BDO being a precursor for aviation fuels and other applications.

### CROSS-REFERENCE TO RELATED APPLICATIONS

This application claims the benefit of U.S. Provisional Application 63/713,253, filed Oct. 29, 2024, the disclosure of which is incorporated by reference in its entirety.

### STATEMENT REGARDING FEDERALLY SPONSORED RESEARCH AND DEVELOPMENT

This invention was made with government support under Contract No. DE-AC05-00OR22725 awarded by the U.S. Department of Energy. The government has certain rights in the invention.

### BACKGROUND OF THE INVENTION

Petroleum-derived carbon-based feedstocks continue to supply a significant portion of the global energy demand. This dependence raises significant economic and environmental concerns, including the depletion of crude oil reserves and the emission of greenhouse gases. To mitigate these issues, there is a growing need for carbon-neutral, renewable energy sources.

One promising route for sustainable fuel production involves the synthesis of 2,3-butanediol (BDO). BDO is a chemical intermediate that can be produced via microbial fermentation from renewable biomass. BDO serves as a precursor for valuable industrial chemicals such as 1,3-butadiene, acetoin, diacetyl, and methyl ethyl ketone, which are used in synthetic rubber and solvents. Further, BDO can be catalytically converted into high-octane hydrocarbons and dioxolanes suitable for use in sustainable aviation fuel formulations.

However, the separation and concentration of BDO remains technically challenging due to its high boiling point, strong affinity for water, and thermal instability at elevated temperatures. Conventional recovery methods, including multi-stage distillation and evaporation, are energy-intensive and may cause BDO degradation or oligomerization. Additionally, impurities present in the fermentation can negatively impact downstream catalysts used for BDO upgrading.

Accordingly, there exists a need for improved methods and systems for the efficient recovery, purification, and catalytic upgrading of bio-derived BDO to enable its use as a renewable feedstock for the production of fuels, including sustainable aviation fuels and other chemicals.

### SUMMARY OF THE INVENTION

In one aspect, the present invention provides a method for the recovery of BDO from a fermentation broth. The method includes directing an aqueous phase along the lumen side or the shell side of a plurality of hollow fibers while simultaneously directing an organic phase along the other of the lumen side or the shell side of the plurality of hollow fibers. The aqueous phase includes a fermentation broth containing BDO, water, and impurities, and the organic phase is initially composed entirely of an organic solvent, the organic solvent being immiscible in water and having an affinity for BDO. The organic solvent continuously recovers BDO from the fermentation broth, the BDO being a precursor for aviation fuels and other applications.

In another aspect, the present invention provides a system for the recovery of BDO from a fermentation broth. The system includes a feed reservoir that holds an aqueous phase comprising the fermentation broth, which contains between 1 wt. % and 20 wt. % of BDO. A membrane module is in fluid communication with the feed reservoir and includes a plurality of hydrophobic hollow fibers. The system also includes a solvent reservoir, which is in fluid communication with the membrane module. The solvent reservoir contains an organic solvent that receives and accumulates the extracted BDO from the membrane module.

In these and other embodiments, the hollow fibers can include hydrophobic materials such as polypropylene (PP), polyvinylidene fluoride (PVDF), polysulfone (PSU), or polytetrafluoroethylene (PTFE). These fibers can have a mean pore size ranging from 10 nm to 1000 nm and a porous sidewall with a thickness between 25 microns and 500 microns. The porous sidewall of the hollow fibers exists as an interface between the aqueous phase and the organic phase to allow contact without phase mixing. Each phase interacts within the pores of the membrane where the transfer of BDO from the fermentation broth to the organic solvent occurs, providing an energy efficient and scalable method for the recovery of high-purity BDO from a fermentation broth.

These and other features and advantages of the present invention will become apparent from the following description of the invention, when viewed in accordance with the accompanying drawings and appended claims.

### BRIEF DESCRIPTION OF THE FIGURES

FIG. 1 illustrates a membrane module for use with a method and a system of the present invention.

FIG. 2 illustrates a system for the recovery of BDO from a fermentation broth in accordance with a method and a system of the present invention.

FIG. 3 is a graph illustrating a laboratory example in which BDO was extracted from a fermentation broth derived from corn stover.

FIG. 4 is a graph illustrating BDO extraction from a simulated fermentation broth at various solvent/feed mass ratios using hexanol as an organic solvent.

FIG. 5 is a graph illustrating BDO extraction from a simulated fermentation broth at various solvent/feed mass ratios using oleyl alcohol as an organic solvent.

FIG. 6 is a graph illustrating BDO extraction rates using PP and PVDF membrane contactors in which hexanol and oleyl alcohol were used an organic solvent.

### DETAILED DESCRIPTION OF THE CURRENT EMBODIMENTS

The invention as contemplated and disclosed herein includes methods and systems for the recovery of BDO from a fermentation broth. In general terms, the method includes the following steps: (a) providing a membrane module including a plurality of porous hollow fibers; (b) applying a continuous flow rate of an aqueous phase (containing BDO) along the lumen side or the shell side of the plurality of porous hollow fibers; and (c) applying a continuous flow rate of an organic phase along the other of the lumen side or the shell side of the plurality of porous hollow fibers, the organic phase including an organic solvent that is immiscible and water and has an affinity for BDO. The steps of applying a flow rate of an aqueous phase and a flow rate of an organic phase are generally simultaneous. These steps are discussed below in connection with the recovery of BDO from a fermentation broth, optionally as a precursor for aviation fuels.

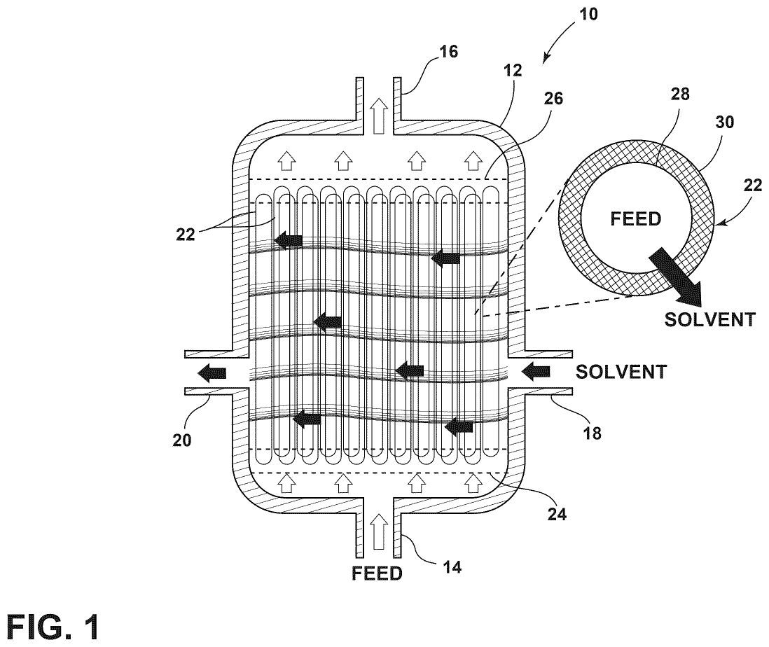

At step (a), providing a membrane module generally includes providing a plurality of hollow or tube-like fibers extending between opposing tubesheets. One example of a membrane module is illustrated in FIG. 1 and generally designated 10. The membrane module 10 includes an outer casing 12 including a feed input port 14, a feed output port 16, a solvent input port 18, and a solvent output port 20. The plurality of hollow fibers 22 are potted to first and second tubesheets 24, 26 at opposing ends thereof, such that the fibers 22 extend in a common direction. Each fiber 22 includes a lumen side 28 and a shell side 30. The lumen side 28 is illustrated in FIG. 1 as being exposed to the aqueous phase, however in other embodiments the lumen side 28 is exposed to the organic phase. Similarly, the shell side 30 is illustrated in FIG. 1 as being exposed to the organic phase, however in other embodiments the shell side 30 is exposed to the aqueous phase.

The hollow fibers 22 can be formed from a hydrophobic material, for example polypropylene (PP), polyvinylidene fluoride (PVDF), polysulfone (PSU), or polytetrafluoroethylene (PTFE). The hollow fibers include a mean pore size of between 10 nm and 1000 nm, optionally between 10 nm and 100 nm, further optionally between 30 nm and 40 nm. The hollow fibers have a mean sidewall thickness of between 25 microns and 500 microns, further optionally between 50 microns and 100 microns, still further optionally between 200 microns and 400 microns. The present invention is not strictly limited to hydrophobic fibers, as other embodiments can include hydrophilic fibers.

Step (b) includes directing a continuous flow rate of an aqueous phase from a feed reservoir along the lumen side or the shell side of the plurality of porous hollow fibers. As shown in FIG. 1, the aqueous phase can be directed through the module 10 along the lumen side 28 of each of the plurality of porous hollow fibers 22. Alternatively, the aqueous phase can be directed through the module 10 along the shell side 30 of each of the plurality of fibers 22. The aqueous phase includes water and between 1 wt % and 20 wt % of BDO. The aqueous phase also includes impurities (those typically found in fermentation broths) that are rejected by the organic solvent.

Step (c) includes directing a continuous flow rate of an organic phase along the lumen side or the shell side of the plurality of hollow fibers. The organic phase includes the organic solvent and the recovered BDO at concentrations that increase linearly over time. The organic solvent is immiscible in water, for example hexanol, oleyl alcohol, or isobutanol. Other organic solvents can be used in other embodiments where desired. The organic phase is directed through the module 10 along the shell side 30 of each of the plurality of fibers 22 as shown in FIG. 1, optionally in a direction generally transverse to the flow of the feed solution within the fibers 22. Alternatively, the organic phase can be directed through the interior of the hollow fibers 22 to contact the lumen side 28 thereof. Steps (b) and (c) can be performed continuously until a concentration of BDO in the aqueous phase falls below a predetermined level.

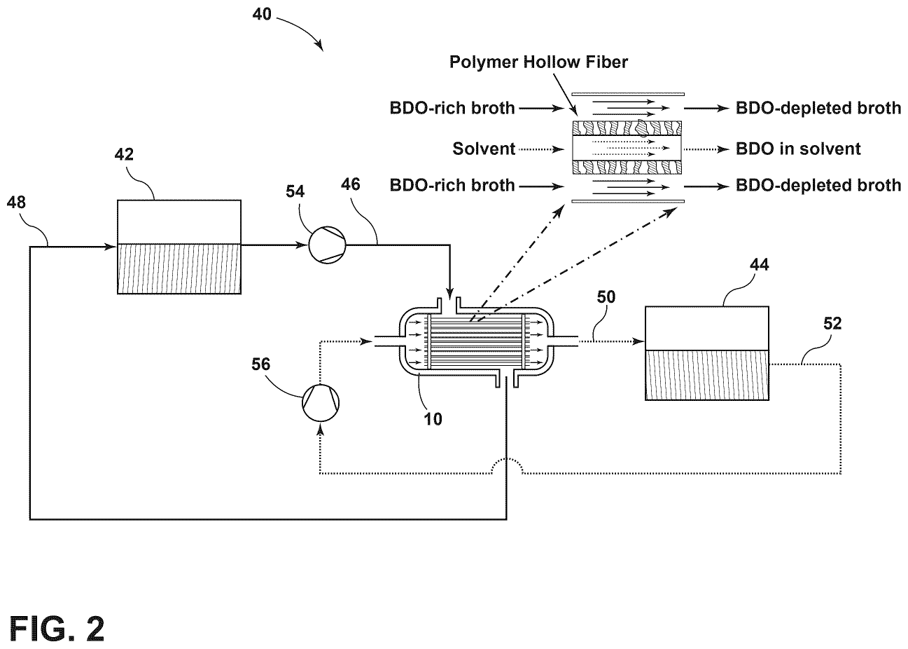

To further illustrate the circulation of the aqueous phase and the organic phase, a system is illustrated in FIG. 2 and generally designated 40. The system 40 includes a feed reservoir 42, a solvent reservoir 44, a membrane module 10, a feed line 46, a feed return line 48, a solvent line 50, and a solvent return line 52. The aqueous phase is contained within the feed reservoir 42. The feed line 46 includes a pump 54, for example a peristaltic pump, to ensure the feed line pressure is slightly greater than the solvent line pressure. The solvent return line 52 also includes a pump 56, for example a peristaltic pump, to ensure a continuous flow of organic phase through the module 10. The aqueous phase and the organic phase are in continuous recirculation, while in other embodiments the feed line and/or the solvent line form an open circuit.

The subsequent separation of BDO from the solvent reservoir 44 can be performed according to any suitable method, including wiped film evaporation or distillation for example. The porous sidewall of the hollow fibers provides a diffusion path from the aqueous phase to the organic phase. In embodiments using hydrophobic fibers, the organic solvent occupies the pores of the hollow fibers. The organic solvent substantial rejects water and impurities in the fermentation broth, while transporting BDO to the solvent interface.

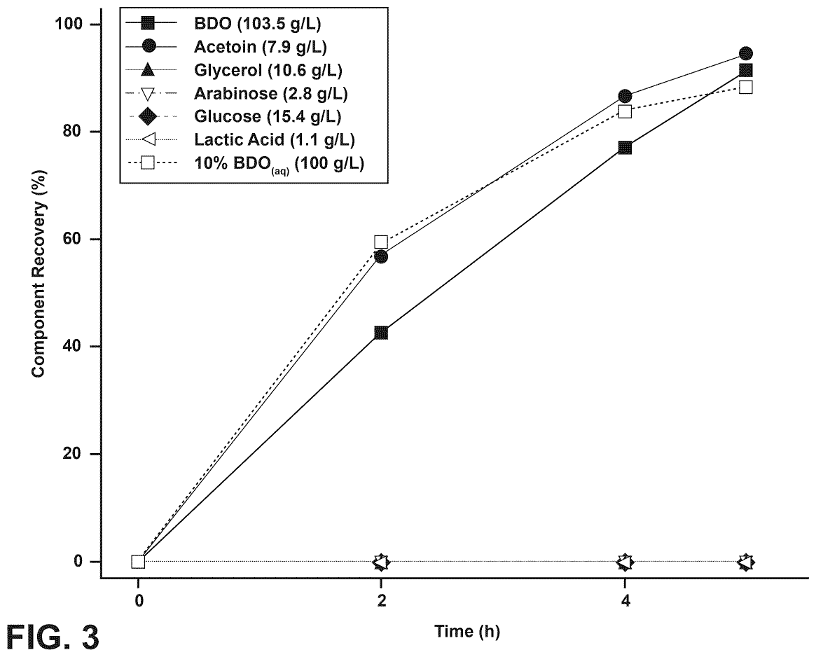

FIG. 3 is a graph illustrating a laboratory example in which BDO was extracted from a fermentation broth derived from corn stover. The organic solvent was hexanol, and greater than 90% of BDO was recovered after a runtime of only 5 hours. Because hexanol has a strong affinity with BDO, hexanol (as the organic solvent) bonds to BDO at the feed interface, and the BDO then diffuses through the porous sidewall into the organic phase. Because the contact between the solvent and the fermentation broth is brief (much shorter than a normal liquid-liquid extraction process), only BDO transfers efficiently into the organic phase for recovery in the organic phase.

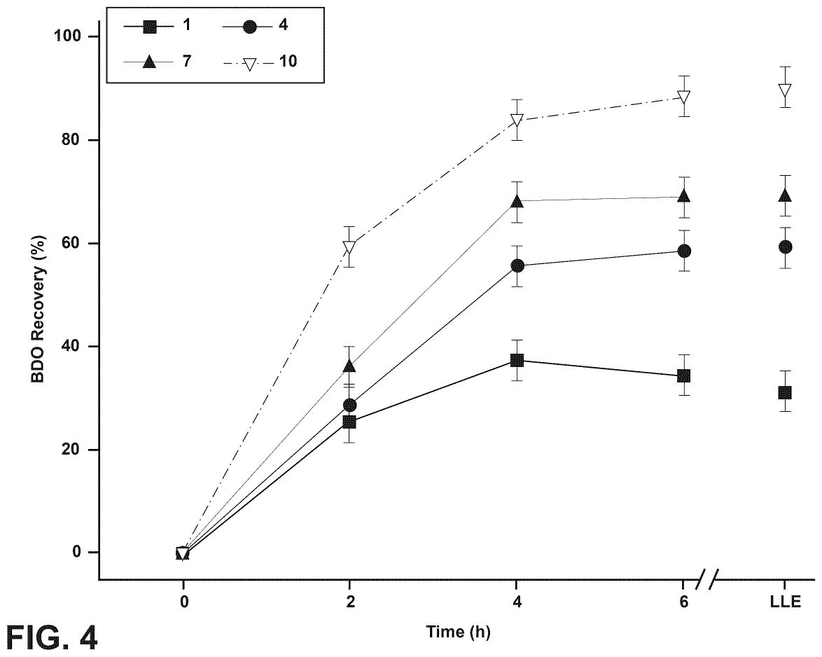

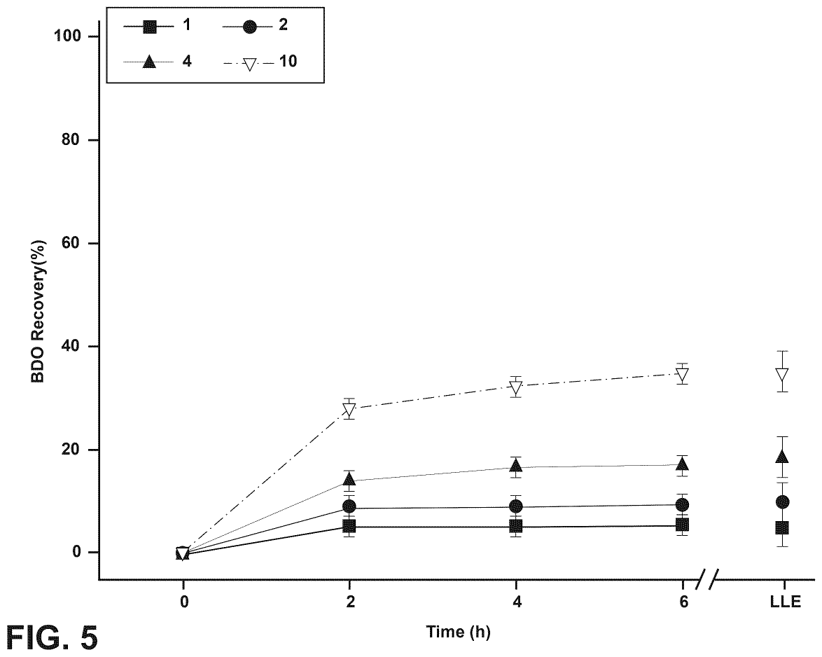

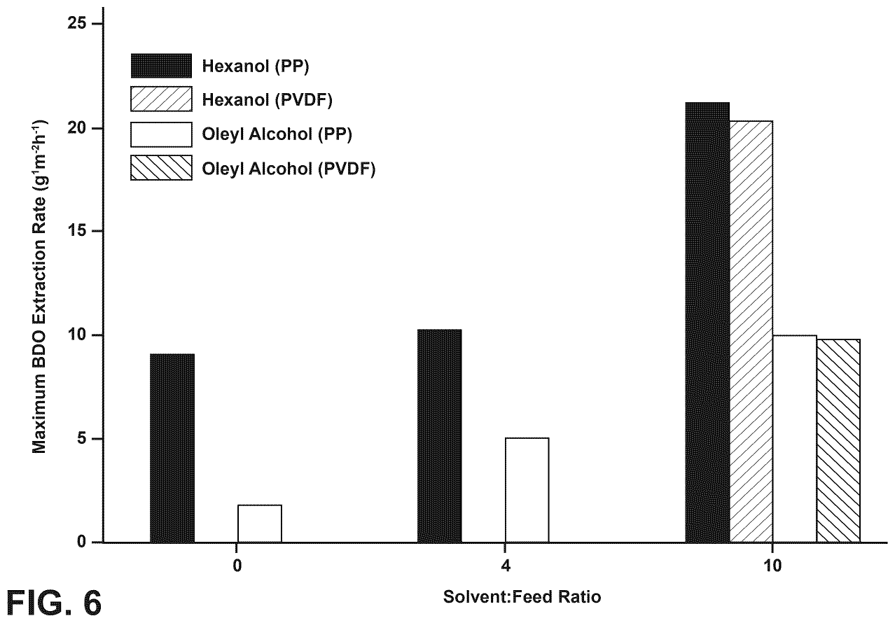

FIG. 4 is a graph illustrating BDO extraction from a simulated fermentation broth containing 10 wt. % BDO at various solvent/feed mass ratios (1, 4, 7, 10). The percent recovery with hexanol as the organic solvent increased linearly for a runtime of approximately four hours, after which the BDO recovery reached equilibrium. Similarly, FIG. 5 is a graph illustrating BDO extraction from a simulated fermentation broth containing 10 wt. % BDO at at various solvent/feed mass ratios, but with oleyl alcohol as the organic solvent. In this example, the percent recovery of BDO increased linearly for two hours before reaching equilibrium, which was consistent across a wide range of solvent/feed mass ratios (1, 2, 4, 10). Collectively, these examples indicate that hexanol and oleyl alcohol can both be used as a solvent for BDO recovery from a complex fermentation broth. Oleyl alcohol demonstrated a high extraction rate for BDO from simulated feeds. In contrast, hexanol has a far higher extraction efficiency than oleyl alcohol, and a lower solvent requirement may compensate for the costs associated with downstream solvent regeneration and BDO recovery. Lastly, FIG. 6 compares the performance of PP and PVDF membrane contactors for both hexanol and oleyl alcohol as organic solvents. While the choice of solvent (e.g., hexanol v. oleyl alcohol) can impact BDO extraction rates, the choice of hydrophobic fibers (e.g., PP v. PVDF) did not have a significant impact on extraction performance, particularly for a 10:1 solvent-to-feed ratio.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims.

1. A method for the separation and recovery of 2,3-butanediol (BDO) from a fermentation broth, the method comprising:
providing a plurality of hollow fibers, each of the plurality of hollow fibers being hydrophobic and including a porous polymeric sidewall defining a lumen side and a shell side; and
directing an aqueous phase containing the fermentation broth along one of the lumen side or the shell side of the plurality of hollow fibers while simultaneously directing an organic phase containing an organic solvent along the other of the lumen side or the shell side of the plurality of hollow fibers to continuously recover BDO from the fermentation broth across the porous polymeric sidewall, the organic solvent being immiscible in water.

2. The method of claim 1, wherein the organic solvent includes hexanol, oleyl alcohol, or isobutanol.

3. The method of claim 1, wherein the plurality of hollow fibers include polypropylene (PP), polyvinylidene fluoride (PVDF), polysulfone (PSU), or polytetrafluoroethylene (PTFE).

4. The method of claim 1, wherein the porous sidewall of the plurality of hollow fibers defines a mean pore size of between 10 nm and 1000 nm.

5. The method of claim 1, wherein the porous sidewall of the plurality of hollow fibers defines a thickness of between 25 microns and 500 microns.

6. The method of claim 1, wherein the fermentation broth comprises between 1 wt. % and 20 wt. % of BDO.

7. The method of claim 1, further comprising maintaining the pH of the aqueous phase to be between 3.0 and 9.0.

8. The method of claim 1, wherein a concentration of BDO in the organic phase increases linearly over time.

9. The method of claim 8, wherein a concentration of BDO in the aqueous phase decreases linearly over time.

10. The method of claim 1, wherein a feed-side pressure is greater than a solvent-side pressure by at least 4 psi.

11. The method of claim 1, further comprising performing wiped film evaporation or distillation to separate BDO from the organic phase.

12. A system comprising:
a feed reservoir including an aqueous phase containing a fermentation broth, the fermentation broth including between 1 wt % and 20 wt % of 2,3-butanediol (BDO);
a membrane module including a plurality of hollow fibers, the plurality of hollow fibers being nanoporous and hydrophobic; and
a solvent reservoir in fluid communication with the membrane module and including an organic phase having an organic solvent that is immiscible in water;
wherein the membrane module recovers BDO from the aqueous phase while substantially rejecting water and impurities contained in the fermentation broth.

13. The system of claim 12, wherein the organic solvent includes hexanol, oleyl alcohol, or isobutanol.

14. The system of claim 12, the plurality of hollow fibers include polypropylene (PP), polyvinylidene fluoride (PVDF), polysulfone (PSU), or polytetrafluoroethylene (PTFE).

15. The system of claim 12, wherein the plurality of hollow fibers define a mean pore size of between 10 nm and 100 nm.

16. The system of claim 12, wherein the porous sidewall of the plurality of hollow fibers defines a thickness of between 25 microns and 500 microns.

17. The system of claim 12, wherein a feed-side pressure is greater than a solvent-side pressure.

18. The system of claim 12, wherein:
the plurality of hollow fibers include polypropylene (PP);
the plurality of hollow fibers define a mean pore size of between 30 nm and 40 nm; and
the plurality of hollow fibers define a thickness of between 50 microns and 100 microns.

19. The system of claim 12, wherein:
the plurality of hollow fibers include polypropylene (PVDF);
the plurality of hollow fibers define a mean pore size of between 10 nm and 100 nm; and
the plurality of hollow fibers define a thickness of between 200 microns and 400 microns.

20. The system of claim 12, wherein the pH of the aqueous phase is between 3.0 and 9.0.
